(19) United States
(12) Patent Application Publication
(10) Pub. No.: US 2023/0110444 A1
(43) Pub. Date: Apr. 13, 2023

(54) UNDERPAD WITH WETNESS INDICATOR

(71) Applicant: Medline Industries, LP, Northfield, IL (US)

(72) Inventors: Alyssa Kurt, Kenosha, WI (US); Cole Fuerste, Vernon Hills, IL (US); Morgan Uridil, Cedar Rapids, IA (US); Jessica Iacullo, Chicago, IL (US); Danielle Calverley, Oak Creek, WI (US); Kushal Basnet, Bettendorf, IA (US); Jacob Sessions, Chicago, IL (US)

(21) Appl. No.: 17/500,464

(22) Filed: Oct. 13, 2021

(51) Int. Cl. A61F 13/42; A61F 13/514; A61F 13/513; A61F 13/15

(52) U.S. Cl. CPC A61F 13/42; A61F 13/51496; A61F 13/51394; A61F 2013/422; A61F 2013/15024

(57) ABSTRACT

An underpad for covering a patient support structure includes an upper, patient facing surface and a lower, support structure facing surface. The underpad includes a liquid permeable patient facing layer and a liquid impermeable base layer. An absorbent layer is positioned above the base layer and below the patient facing layer, and a tissue layer is positioned above the absorbent layer and below the patient facing layer. An ink is deposited on the tissue layer. The ink is more readily visible through the patient facing layer than through the base layer.

### FIELD OF THE INVENTION

The present invention relates generally to absorbent articles and, in particular, to an absorbent article for use as an absorbent pad having a wetness indicator.

### BACKGROUND

Bed sores, also known as pressure ulcers or decubitus ulcers, are prevalent among people who are bed-ridden or otherwise immobilized. Skin ulcers can be caused by pressure exerted on the skin and soft tissues (e.g., the individual's body weight resting against a hard surface such as a bed or chair) and are exacerbated when the skin is also exposed to moisture (e.g., due to incontinence) and/or friction, heat, and shear forces, for example caused by moving or repositioning a bed-ridden patient.

Hospital patients are susceptible to various concerns and complications arising from soiled bed pads, including increased risk of infection or contamination. In addition, elderly nursing home residents are particularly vulnerable to pressure ulcers since they are frequently bed-ridden and incontinent. Since pressure ulcers can be persistent and heal slowly, treating pressure ulcers once formed is thus expensive, so there is a significant need to minimize a patient's exposure to conditions which would cause such ulcers.

Accordingly, clinicians need to be able to quickly identify when an absorbent article, such as an underpad, has been soiled and needs to be changed. Often, a clinician must log-roll or otherwise shift the patient to view the pad underneath the patient and look for the presence of bodily fluid. Moving a patient may require more than one clinician and/or may be time-consuming for the clinician. Moving a patient in such a manner also puts the clinician and patient at higher risk of injury.

Therefore, there exists a need for an underpad that easily allow a clinician or caregiver to easily observe when an underpad has been soiled on. Color-changing wetness indicators are known in other incontinence articles including briefs, however, the wetness indicator is printed on the poly backsheet material and viewed from the outside of the brief. In the case of the underpad, printing on the poly backsheet would result in the wetness indicator being obscured by the layers of absorbent material from the top of the underpad (patient-facing side). The wetness indicator could be viewed from the bottom side of the underpad (bed-facing side) but that would require the clinician to lift up the underpad and view the bottom.

Accordingly, it would be advantageous to have an underpad in which the wetness indicator is printed on the underpad such that it is visible from a top, patient-facing side of the underpad.

### BRIEF DESCRIPTION OF THE DRAWINGS

Advantages of the invention will become apparent upon reading the following detailed description and upon reference to the drawings.

FIG. 1 is an exploded cross-section of an absorbent pad in accordance with an embodiment of an underpad.

FIG. 2 is an exploded cross-section of an absorbent pad in accordance with further embodiments of an underpad.

FIG. 3 is a top view of the absorbent pad of FIG. 2.

FIG. 4 is an exploded cross-section of an absorbent pad in accordance with further embodiments of an underpad.

FIG. 5 is an exploded cross-section of an absorbent pad in accordance with further embodiments of an underpad.

FIG. 6 is a top view of an absorbent pad in accordance with further embodiments of an underpad.

FIG. 7 is a top view of an absorbent pad in accordance with further embodiments of an underpad.

While the invention is susceptible to various modifications and alternative forms, specific embodiments have been shown by way of example in the drawings and will be described in detail herein. It should be understood, however, that the invention is not intended to be limited to the particular forms disclosed. Rather, the invention is to cover all modifications, equivalents, and alternatives falling within the spirit and scope of the invention.

### DESCRIPTION OF ILLUSTRATIVE EMBODIMENTS

Embodiments of the invention are now described in detail. Referring to the drawings, like numbers indicate like parts throughout the views. As used in the description herein and throughout the claims, the following terms take the meanings explicitly associated herein, unless the context clearly dictates otherwise: the meaning of “a,” “an,” and “the” includes plural reference, the meaning of “in” includes “in” and “on.” Relational terms such as first and second, top and bottom, forward and rearward, and the like may be used solely to distinguish one entity or action from another entity or action without necessarily requiring or implying any actual such relationship, direction or order between such entities or actions.

In particular, throughout the description, reference is made to top/upper and bottom/lower layers. In general, the upper layer refers to a layer closer to the user as compared to a lower layer, and the lower layer refers to the layer farther from the user as compared to an upper layer. The lower layer may be the layer closer to a support surface on which a user is disposed. However, unless expressly indicated, this convention is only to aid in referencing the various layers and is not intended to limit the scope of the disclosed or claimed embodiments.

As used herein, the following terms have the following meanings:

“Attach” and its derivatives refer to the joining, adhering, connecting, bonding, sewing together, or the like, of two elements. Two elements will be considered to be attached together when they are integral with one another or attached directly to one another or indirectly to one another, such as when each is directly attached to intermediate elements. “Attach” and its derivatives include permanent, releasable, or refastenable attachment. In addition, the attachment can be completed either during the manufacturing process or by the end user.

“Bond” and its derivatives refer to the joining, adhering, connecting, attaching, sewing together, or the like, of two elements. Two elements will be considered to be bonded together when they are bonded directly to one another or indirectly to one another, such as when each is directly bonded to intermediate elements. “Bond” and its derivatives include permanent, releasable, or refastenable bonding.

“Connect” and its derivatives refer to the joining, adhering, bonding, attaching, sewing together, or the like, of two elements. Two elements will be considered to be connected together when they are connected directly to one another or indirectly to one another, such as when each is directly connected to intermediate elements. “Connect” and its derivatives include permanent, releasable, or refastenable connection. In addition, the connecting can be completed either during the manufacturing process or by the end user.

“Integral” is used to refer to various portions of a single unitary element rather than separate structures bonded to or placed with or placed near one another.

These terms may be defined with additional language elsewhere in the specification.

It should be observed that the embodiments reside primarily in the combinations of assembly components and method steps for using various embodiments of the absorbent articles disclosed herein. Accordingly, the assembly components and the method steps have been represented (where appropriate) by conventional symbols in the drawings, showing only those specific details that are pertinent to understanding the embodiments of the present invention so as not to obscure the disclosure with details that will be readily apparent to those of ordinary skill in the art having the benefit of the description herein.

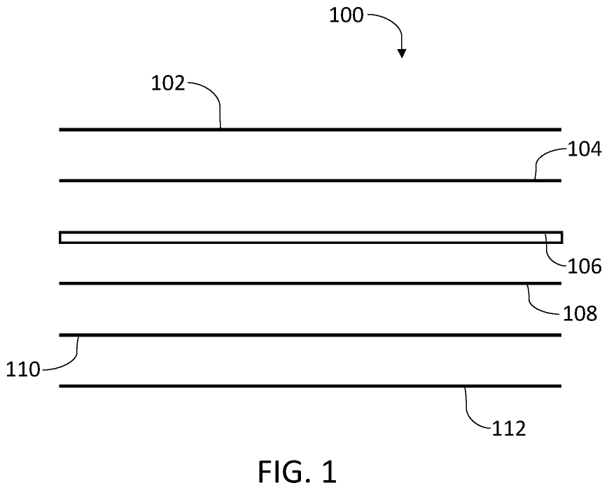

As illustrated in FIG. 1 embodiments of an underpad 100 may comprises a first, patient facing layer 102. This patient facing layer may be a nonwoven material. The nonwoven material of the first layer can be manufactured using any technique known in the art. Non-limiting examples of suitable types of nonwoven materials include staple nonwoven materials, melt-blown nonwoven materials, spunmelt nonwoven materials, spunbond nonwoven materials, SMS (spunbond meltblown spunbond) materials, spun lace materials, needle-felted materials, thermal-bonded nonwoven materials, trough-air-bonded nonwoven material, spunlaid nonwoven material, air-laid nonwoven materials or the like, or any combinations thereof.

In certain embodiments of the invention the first layer is hydrophilic. For example, the first layer can be treated in order make it liquid permeable. Such treatments can include any treatment known in the art which renders a material liquid permeable. Non-limiting examples of such treatments include: coating the surface of the material with a hydrophilic surfactant as described in WO 93/04113 entitled “Method for hydrophilizing absorbent foam materials” and in WO 95/25495 entitled “Fluid acquisition and distribution member for absorbent core”; surface treatments such as corona and plasma treatment as described in described in U.S. Pat. No. 6,118,218 entitled “Steady-state glow-discharge plasma at atmospheric pressure”; applying a hydrophilic coating by a plasma polymerization process as described in WO 00/16913 entitled “Durably wettable, liquid pervious webs” and WO 00/16914 entitled “Durably wettable, liquid pervious webs prepared using a remote plasma polymerization process”; or contacting the fibers with a solution of hydrophilic monomers and radical polymerization initiators and exposing the fibers to UV radiation as described in U.S. Pat. No. 7,521,587 entitled “Absorbent articles comprising hydrophilic nonwoven fabrics”; all of which are hereby incorporated by reference in their entirety for all purposes.

In another embodiment of the invention, the first layer is permeable to liquids such that the liquids can pass through the surface of the first layer toward the second layer (disposed beneath the first layer), but the liquids cannot substantially reverse direction and move back toward the surface of the first layer. In other words, in various embodiments the flow of liquids through the first layer is substantially unidirectional from the top surface of the first layer toward the second layer disposed beneath the first layer.

The first layer can comprise any suitable material known in the art. For example, the first layer can comprise a polymeric material. Non limiting examples of such polymeric materials include polypropylene, polyethylene, polyethylene terephthalate, polyamide, viscose rayon, nylon, or the like or any combinations thereof. Furthermore, the polymeric material can be a biodegradable polymeric material. One such non-limiting example of a polymer is the starch-based biodegradable material described in United States Patent Application 2009/0075346, which is hereby incorporated by reference for all purposes. In certain embodiments of the invention, the first layer has a weight per unit area (weight) in the range of about 15 gsm (grams per square meter) to about 20 gsm. For example, the first layer can have a weight of about 15 gsm, about 16 gsm, about 17 gsm, about 18 gsm, about 19 gsm, or about 20 gsm, inclusive of all ranges and subranges therebetween.

In certain embodiments of the invention, the first layer is fluid permeable. In one embodiment of the invention the first layer has a Moisture Transfer Rate, for example as measured by ASTM E96M-05 in the range of about 5 to about 200 sec/mL.

In another embodiment of the invention, the first layer is air permeable. In certain embodiments, the first layer has an air permeability of the range of about 10 seconds/100 mL to about 100 seconds/100 mL, including about 10 seconds/100 mL, about 20 seconds/100 mL, about 30 seconds/100 mL, about 40 seconds/100 mL, about 50 seconds/100 mL, about 60 seconds/100 mL, about 70 seconds/100 mL, about 80 seconds/100 mL, about 90 seconds/100 mL, or about 100 seconds/100 mL, inclusive of all ranges and subranges therebetween, for example as measured by the Gurely method using a densometer, (e.g., methods conforming the following standards: ASTM D737 and WSP 70.1)

A second, tissue layer 104 may be positioned below the patient facing, non-woven layer 102. The second layer may comprise a tissue layer. The tissue layer may comprise a tissue paper formed from a paper pulp comprising various appropriate materials, including for example northern softwood, eucalyptus and water, among other materials. Alternatively, the tissue paper may be formed from a pulp using recovered fibers such as a pulp comprising recycled paper or other fabric material. The tissue layer may be formed of any liquid permeable material that may be printed on as described below. Appropriate materials include tissue paper, but may also include other non-woven materials as would be understood by one of ordinary skill in the art.

The tissue paper may also be produced utilizing a wet or dry crepe operation. Embodiments of the tissue paper may also be produced utilizing a through-air-drying operation. Other processes and materials known in the art may be used in further embodiments. The tissue layer may have a weight per unit area (weight) in the range of about 10 gsm (grams per square meter) to about 60 gsm. More preferably, the tissue layer may have a weight per unit area in the range of about 12 gsm to about 17 gsm. For example, the tissue layer can have a weight of about 12 gsm, about 13 gsm, about 14 gsm, about 15 gsm, about 16 gsm, or about 17 gsm, inclusive of all ranges and subranges therebetween.

A third, absorbent layer 106 may be positioned below the second, tissue layer 104. In certain embodiments the third layer is comprised of a formed material. The formed material of the third layer can be manufactured using any technique known in the art. Non-limiting examples of suitable types of formed materials include staple nonwoven materials, melt-blown nonwoven materials, spun-melt nonwoven materials, spun-bound nonwoven materials, thermal-bonded nonwoven materials, trough-air-bonded nonwoven materials, spun-laid nonwoven materials, air-laid nonwoven materials, or any combinations thereof. In a particular embodiment the third layer is comprised of an air-laid fiber. In one embodiment the air-laid fiber is thermobonded. In a particular embodiment the air-laid material is air laid paper.

The third layer can comprise fibers, for example natural fibers. The natural fibers can be any suitable natural fibers known in the art. In one embodiment the natural fiber is cellulose. The cellulose can be from any suitable source known in the art. Non-limiting examples of suitable sources of cellulose are wood fibers, plant fibers, field crop fibers, fluff pulp fibers, cotton, any other material, man-made or natural, designed to absorb liquid, or any combination thereof. In a particular embodiment the second layer comprises wood fibers. In another embodiment, the second layer comprises macerated wood pulp.

The third layer of the pad can further comprise an absorbent polymer, for example any super-absorbent polymer known in the art. Non-limiting examples of suitable super-absorbent polymers include, for example, polymers and copolymers of acrylic acid and salts thereof (including alkali metal salts such as sodium salts, or alkaline earth salts thereof), polymers and copolymers of methacrylic acid and salts thereof (including alkali metal salts such as sodium salts, or alkaline earth salts thereof), polyacrylamide polymers and copolymers, ethylene maleic anhydride copolymers, cross-linked carboxy-methyl-celluloses, polyacrylate/polyacrylamide copolymers, polyvinyl alcohol copolymers, cross-linked polyethylene oxides, starch grafted copolymers of polyacrylonitrile, etc. The super-absorbent polymers can be cross-linked to suitable degree.

In a particular embodiment the super-absorbent polymer comprises sodium polyacrylate. In another embodiment, the third layer comprises an amount of super-absorbent polymer(s) in the range of about 15 gsm to about 35 gsm. For example, the super-absorbent polymer(s) in the second layer is present in an amount of about 15 gsm, about 20 gsm, about 25 gsm, about 30 gsm, or about 35 gsm, inclusive of all ranges and subranges therebetween.

In another embodiment, the cellulose fibers of the third layer are present in the third layer in an amount of about 85 gsm to about 115 gsm. For example, the cellulose fibers of the third layer are present in an amount of about 85 gsm, about 90 gsm, about 95 gsm, about 100 gsm, about 103 gsm, about 105 gsm, about 110 gsm, about 115 gsm, or about 120 gsm, inclusive of all ranges and subranges therebetween.

In a particular embodiment, the third layer is a thermobonded, absorbent airlaid core made from cellulose fibers and super-absorbent polymers. In a particular embodiment of the invention, the third layer is comprised of an airlaid absorbent core as described in U.S. Pat. No. 6,675,702 which is hereby incorporated herein by reference for all purposes. In yet another embodiment, the third layer is comprised of a thermobonded airlaid core made from about 100 to about 105 gsm of cellulose fibers and 25 gsm of super absorbent polymers. In a particular embodiment, the cellulose fibers are macerated wood pulp.

The third layer absorbs substantially all of the liquids penetrating through from the first and second layer, and has a liquid-holding capacity sufficient to retain liquids without releasing the liquid through the first and second layers. In certain embodiments, the third layer has an absorption capacity in the range of about 50 cc/m2 to about 20,000 cc/m2, for example, about 50 cc/m2, about 100 cc/m2, about 150 cc/m2, about 200 cc/m2, about 250 cc/m2, about 300 cc/m2, about 350 cc/m2, about 400 cc/m2, about 450 cc/m2, about 500 cc/m2, about 550 cc/m2, about 600 cc/m2, about 650 cc/m2, about 700 cc/m2, about 750 cc/m2, about 800 cc/m2, about 850 cc/m2, about 900 cc/m2, about 1,000 cc/m2, about 1,100 cc/m2, about 1,200 cc/m2, about 1,300 cc/m2, about 1,400 cc/m2, about 1,500 cc/m2, about 1,600 cc/m2, about 1,700 cc/m2, about 1,800 cc/m2, about 1,900 cc/m2, about 2,000 cc/m2, about 3,000 cc/m2, about 4,000 cc/m2, about 5,000 cc/m2, about 6,000 cc/m2, about 7,000 cc/m2, about 8,000 cc/m2, about 9,000 cc/m2, about 10,000 cc/m2, about 11,000 cc/m2, about 12,000 cc/m2, about 13,000 cc/m2, about 14,000 cc/m2, about, 15,000 cc/m2, about 16,000 cc/m2, about 17,000 cc/m2, about, 18,000 cc/m2, about 19,000 cc/m2, or about 20,000 cc/m2 inclusive all ranges and subranges therebetween, as measured by the ISO11948-1 test method.

The pad 100 may further comprise a fourth layer 108. The fourth layer may comprise a tissue layer using the same material set forth above with regard to the second layer 104.

The pad 100 may further comprise a fifth layer 110. This layer may also be referred to as the base layer. The fifth layer is not liquid permeable and is intended to capture liquid absorbed by the pad and not allow the liquid to pass through the pad to the patient supporting structure beneath. In particular, the fifth layer 110 may comprise a polypropylene material that does not allow passage of liquid.

The base layer prevents the liquid absorbed in the upper layers to penetrate through the base layer of the absorbent pad (or in some embodiments, the fourth layer). The base layer can comprise any natural or man-made material capable of preventing the flow of liquids out of the upper layers and through the bottom of the absorbent pad. In certain embodiments wherein the base layer comprises a polymeric film, for example a hydrophobic polymeric film. The polymeric film of the base layer can be any suitable polymer known in the art, for example suitable hydrophobic polymers. Non-limiting examples of such polymers include polyolefins such as polyethylene, polypropylene, poly(lactic acid), polyhydroxybutyrate, and tapioca starch as well as copolymers thereof. One such non-limiting example of a polymer is the starch-based biodegradable material described in United States Patent Application 2009/0075346, which is hereby incorporated herein by reference for all purposes.

The base layer should also provide for air circulation within the absorbent pad to prevent heat and moisture vapor build up. Accordingly, in particular embodiments, the base layer is air permeable. Air permeability can be provided in various ways, for example by forming a base layer comprising a woven or nonwoven hydrophobic material which prevents the movement of bulk liquid, but allows diffusion or movement of air through the third layer. For example, the hydrophobic material can comprise hydrophobic polymeric fibers (e.g., polyolefin fibers) or comprising fibers surface treated with a hydrophobic sizing or coating. In yet another embodiment the base layer comprises a perforated polyolefin (e.g. polyethylene and/or polypropylene polymer or copolymer) sheet. If the base layer comprises a perforated polyolefin sheet, the perforations should be of a size which does not permit the permeation or movement of liquids through the perforations, but does provide air permeability values within the ranges described herein.

Although the base layer does not permit any appreciable amount of liquid to flow through, in many instances it can be advantageous to allow moisture vapor to permeate through the base layer. In certain embodiments of the invention, the base layer has a moisture vapor transmission rate (MTVR) in the range of about 1,000 g/m2/day to about 10,000 g/m2/day. For example, the base layer can have an MTVR of about 1,000 g/m2/day, about 2,000 g/m2/day, about 3,000 g/m2/day, about 4,000 g/m2/day, about 5,000 g/m2/day, about 6,000 g/m2/day, about 7,000 g/m2/day, about 8,000 g/m2/day, about 9,000 g/m2/day, or about 10,000 g/m2/day, inclusive of all ranges and subranges therebetween.

In yet another embodiment of the invention, the base layer has a moisture vapor transmission rate (MTVR) in the range of about 2,500 g/m2/day to about 4,500 g/m2/day. For example, the base layer can have an MTVR of about 2,500 g/m2/day, about 2,600 g/m2/day, about 2,700 g/m2/day, about 2,800 g/m2/day, about 2,900 g/m2/day, about 3,000 g/m2/day, about 3,100 g/m2/day, about 3,200 g/m2/day, about 3,300 g/m2/day, about 3,400 g/m2/day, about 3,500 g/m2/day, about 3,600 g/m2/day, about 3,700 g/m2/day, about 3,800 g/m2/day, about 3,900 g/m2/day, about 4,000 g/m2/day, about 4,100 g/m2/day, about 4,200 g/m2/day, about 4,300 g/m2/day, about 4,400 g/m2/day or about 4,500 g/m2/day, inclusive of all ranges and subranges therebetween.

In various embodiments of the present invention, the base layer has a weight in the range of about 20 gsm to about 45 gsm. For example, the base layer can have a weight of about 20 gsm, 25 gsm, 30 gsm, 35 gsm, 40 gsm, or 45 gsm inclusive of all ranges and subranges therebetween.

Finally, the underpad 100 may comprise a sixth, outer layer 112. The outer layer 112 may be a non-woven layer and may have the same material and/or characteristics as the patient-facing, top layer 102.

In various embodiments, the layers are adhered together. The layers can be adhered together using any suitable technique known in the art. In a particular embodiment, the layers are adhered together using an adhesive. Any suitable adhesive known in the art can be used. The adhesive used can be natural or synthetic. Non-limiting examples of such adhesives are hot melt adhesives, drying adhesives, contact adhesives, UV curing adhesives, light curing adhesives, and pressure sensitive adhesives or the like. In one embodiment, the top layer and the edges where the layers meet are glued together using hot melt adhesive.

Non-limiting examples of adhesives include animal glue, collagen-based glue, albumin glue, casein glue, Canada balsam, coccoina, pelikanol, gum Arabic, latex, methyl cellulose, library glue, mucilage, resorcinol resin, starch, urea-formaldehyde resin, acrylonitrile, cyanoacrylate, acrylic, epoxy resins, epoxy putty, ethylene-vinyl acetate, phenol formaldehyde resin, polyamide, polyester resins, polyethylene, polypropylene, polysulfides, polyurethane, polyvinyl acetate, polyvinyl alcohol, polyvinyl chloride, polyvinyl chloride emulsion, polyvinylpyrrolidone, rubber cement, silicones, styrene acrylic copolymer, ethylene-acrylate copolymers, polyolefins, atactic polypropylene, polybutene-1, oxidized polyethylene, styrene block copolymers, polycarbonates, fluoropolymers, silicone rubbers, or the like and various other co-polymers. The adhesive may further comprise one or more additives. Any suitable additive known in the art can be used. Non-limiting examples of additives include, tackifying resins, waxes, plasticizers, antioxidants, stabilizers, UV stabilizers, pigments, dyes, biocides, flame retardants, antistatic agents, and fillers or the like. In particular embodiments, the adhesive comprises a hot-melt adhesive.

The adhesive layer(s) can be continuous, contacting substantially the entire surface area of any two layers adhered together (e.g. at least about 75%, at least about 80%, at least about 85%, at least about 90%, at least about 90%, or essentially about 100% of the surface area of the two layers adhered together, inclusive of all ranges and subranges therebetween). That is, the adhesive forms an intermediate layer between any two layers adhered together, contacting substantially the entire surfaces between the two layers. Alternatively, the adhesive can be applied in a pattern (e.g., grid) or random fashion whereby the adhesive does not substantially contact the entire surface area of the two layers, but rather forms a discontinuous intermediate layer between the two adhered surfaces. Each of the first, second, and base layers (or first, second, third, and fourth layers when present) of the absorbent pad of the present invention can be adhered together with continuous adhesive layers, or with discontinuous adhesive layers, or some of the adhesive layers can be continuous adhesive layers, and other adhesive layers can be discontinuous layers. Each of the adhesive layers can comprise the same adhesive material (as described herein), or one or more of the adhesive layers can comprise a different adhesive material (as described herein).

The present invention can be assembled from the component layers by any suitable method known in the art. In one embodiment of the invention, rolls of each layer are combined such that each layer is disposed over the preceding layer, and the combined layers are then cut to the appropriate size and the edges adhered together. In another embodiment of the invention, sheets of a suitable size are cut from rolls of each individual layer, then combined in the appropriate order and the edges are adhered together.

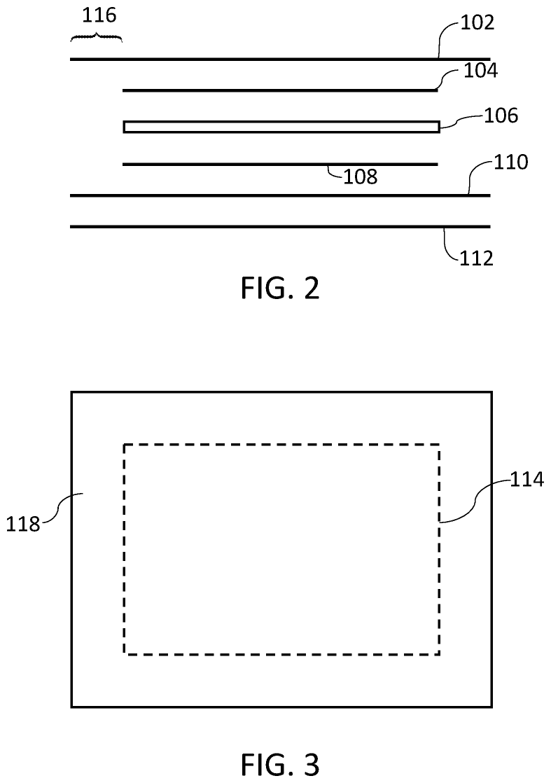

As illustrated in FIG. 1, the layers may be generally coextensive. Alternatively, as illustrated in FIGS. 2-3, the top 102, base 110 and outer 112 layers may extend beyond a peripheral edge 114 of the tissue 104, 108 and absorbent 106 layers. This extension 116 of the top, base and outer layers may create a frame 118 around the absorbent portion of the pad. In such embodiments, the top layer 102 and base layer 110 may be directly adhered to one another in this frame 118 section.

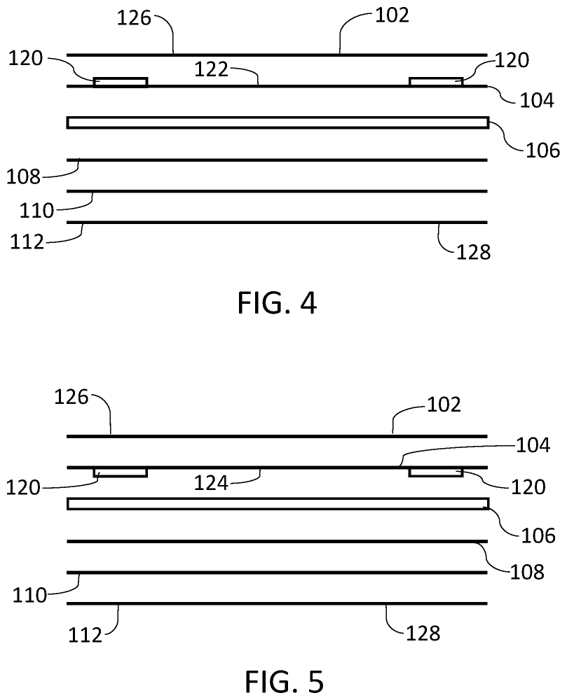

In embodiments of the invention, as illustrated in FIG. 4, a pattern of wetness indicator ink 120 may be printed on a top surface 122 of the tissue layer 104. Alternatively, as illustrated in FIG. 5, the wetness indicator ink 120 may be printed on a bottom surface 124 of the tissue layer 104.

The wetness indicator ink 120 may be a water soluble dye, and in particular, a water soluble dye in a water soluble polyvinyl binder. Embodiments of the wetness indicator comprise a hydrochromatic ink that is printed on the top tissue layer. The hydrochromatic ink is printed in color and once liquid comes in contact with the printed ink the color disappears. This change is irreversible and the color does not reappear when the wetness indicator/ink dries. The underpad is designed to pull liquid away from the top layer and patient and be dry to the touch. In embodiments of the underpad, the color disappears when wet and therefore prevents bleeding of colored ink onto the skin of the patient or linens such as bed sheets or hospital gowns as may occur in inks that appear or change color when contacted by liquid.

A large variety of water-dispersible or water-soluble coloring agents may be used. The criteria are that these coloring agents are capable or ready dissolution or dispersion in aqueous liquids; that the agents have enough color intensity to be readily visible through a light-transmitting transparent or translucent top layer; and that the agent be nontoxic and non-irritating should it inadvertently contact the skin. Various coloring agents which meet these criteria include acid, basic, and direct dyes; soluble inorganic pigments; food and vegetable colors; and the like may be used as would be understood by one of ordinary skill in the art.

In various embodiments, because the top, non-woven layer 102 is relatively transparent or translucent, indicator ink is readily visible from the top, patient-facing surface 126 of the underpad 100. Embodiments of the top layer 102 may have an opacity of less than 0.85 using a diffuse opacity method (R₀/R_{∞}). In further embodiments, the top layer may have an opacity in the range of 0.20 to 0.80, and in yet further embodiments, the top layer may have an opacity in the range of 0.25 to 0.50. If the ink 120 is printed on a lower surface 124 of the tissue layer 104, the tissue layer may also have an opacity that allows the ink to be visible. In such embodiments, the combination of the top layer and the tissue layer may have an opacity of less than 0.85. In further embodiments, the combination of top layer and tissue may have an opacity in the range of 0.20 to 0.80, and in yet further embodiments, the combination may have an opacity in the range of 0.25 to 0.50.

In embodiments of the underpad because of the intervening layers, the indicator ink is less visible from a bottom surface 128 of the underpad. In further embodiments, the indicator ink is not readily visible or not visible at all from a bottom surface 128 of the underpad. In embodiments of the underpad, the combination of layers below the tissue layer may have an opacity that is greater than 0.50. In further embodiments of the underpad, the combination of layers below the tissue layer may have an opacity that is greater than 0.80. In further embodiments of the underpad, the combination of layers below the tissue layer may have an opacity that is greater than 0.90.

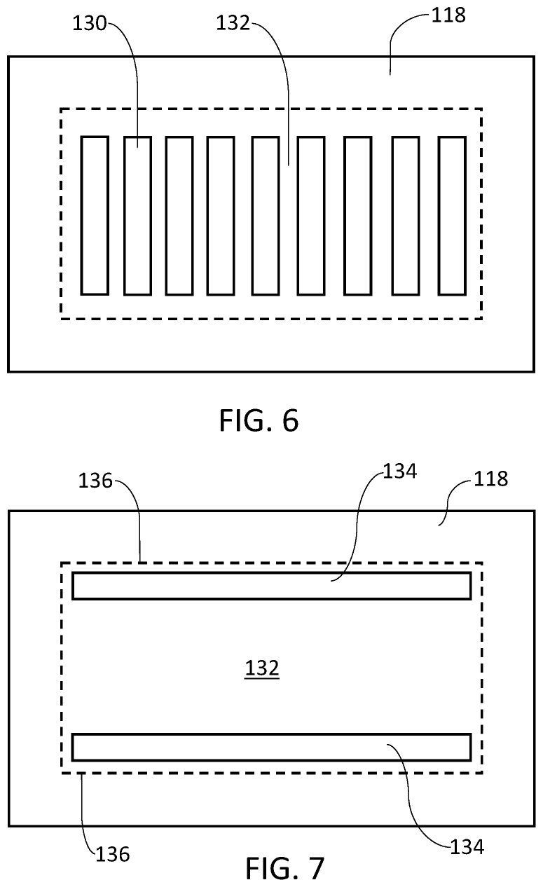

The ink 120 may be arranged in a number of different patterns depending on the patient or practitioner needs or in response to cost considerations. As illustrated in FIG. 6, the wetness ink may be arranged in a number of transverse stripes 130 across the underpad 100. Any apparent break in one the stripes 130 would indicate to a practitioner that the pad had been wetted and may need to be changed. In particular, the stripes 130 may extend across a central portion 132 including the absorbent and tissue layers within the frame 118 created around this central portion.

Alternatively, as illustrated in FIG. 7, the ink 120 may be arranged in one or more longitudinal stripes 134 that parallel an edge 136 of central portion 132. In this manner, any break in longitudinal stripes 134 would indicate to a practitioner that the pad had been wetted to an edge of the absorbent central portion 132. Such a configuration may also have the advantage that the longitudinal edge stripes 134 would be visible to the side of the patient and would not require the practitioner to move the patient in order to observe the status of the ink.

While the present invention has been described with reference to one or more particular embodiments, those skilled in the art will recognize that many changes may be made thereto without departing from the spirit and scope of the present invention. Furthermore, components from one embodiment can be used in other non-exclusive embodiments. By way of example, any of the absorbent articles described herein can include any of the absorbent structures described herein in relation to FIGS. 7-16, and in particular, the absorbent article 100 may include an absorbent structure having any of the various configurations shown in FIG. 16. Each of these embodiments and obvious variations thereof is contemplated as falling within the spirit and scope of the invention, which is set forth in the following alternative embodiments.

What is claimed is:

1. An underpad for covering a patient support structure, the underpad comprising an upper, patient facing surface and a lower, support structure facing surface, the underpad further comprising:
a patient facing layer comprising a liquid permeable material, wherein the patient facing layer forms at least a portion of the underpad upper surface;
an upper tissue layer comprising a liquid permeable material, the upper tissue layer being disposed adjacent to and below a lower surface of the patient facing layer;
an absorbent layer comprising a liquid absorbing material, the absorbent layer being disposed adjacent to and below a lower surface of the tissue layer;
a base layer comprising a liquid impermeable material, the base layer being disposed below a lower surface of the absorbent layer and forming at least a portion of the underpad lower surface; and
a dye deposited on a surface of the upper tissue layer;
wherein the dye is visible through the patient facing layer from the underpad upper surface prior to the dye being exposed to a liquid;
wherein the dye is less visible from the underpad lower surface; and
wherein a change in the dye is visually perceptible through the patient facing layer from the underpad upper surface subsequent to the dye being exposed to a liquid.

2. The underpad of claim 1 further comprising a lower tissue layer comprising a liquid permeable material, the lower tissue layer being disposed adjacent to and below a lower surface of the absorbent layer.

3. The underpad of claim 1 wherein the dye is not visible through the base layer from the underpad lower surface.

4. The underpad of claim 1 wherein the dye comprises a water soluble dye that visually disappears upon dissolution of the dye subsequent to the dye being exposed to a liquid.

5. The underpad of claim 1 wherein the dye comprises a water dispersible dye that visually disappears upon dispersion of the dye subsequent to the dye being exposed to a liquid.

6. The underpad of claim 1 wherein the dye comprises a dye that visually changes color subsequent to the dye being exposed to a liquid.

7. The underpad of claim 1 wherein the dye is printed on the tissue layer prior to assembly of the underpad.

8. The underpad of claim 1 wherein the dye is printed on an upper surface of the tissue layer.

9. The underpad of claim 8 wherein the upper surface of the tissue layer is adjacent to a lower surface of the patient facing layer, and the dye is positioned between the upper surface of the tissue layer and the lower surface of the patient facing layer.

10. The underpad of claim 1 wherein the dye is printed on a lower surface of the tissue layer.

11. The underpad of claim 10 wherein the lower surface of the tissue layer is adjacent to an upper surface of the absorbent layer, and the dye is positioned between the lower surface of the tissue layer and the upper surface of the absorbent layer.

12. The underpad of claim 1 wherein the patient facing layer has an opacity of less than 0.80 using a diffuse opacity method (R₀/R_{∞}).

13. The underpad of claim 12 wherein the patient facing layer has an opacity of less than 0.70 using a diffuse opacity method (R₀/R_{∞}).

14. The underpad of claim 12 wherein the patient facing layer has an opacity of less than 0.50 using a diffuse opacity method (R₀/R_{∞}).

15. The underpad of claim 12 wherein the absorbent layer has an opacity of greater than 0.80 using a diffuse opacity method (R₀/R_{∞}).

16. The underpad of claim 12 wherein the base layer has an opacity of greater than 0.80 using a diffuse opacity method (R₀/R_{∞}).

17. An underpad for covering a patient support structure, the underpad comprising an upper, patient facing surface and a lower, support structure facing surface, the underpad further comprising:
a patient facing layer comprising a liquid permeable material, wherein the patient facing layer forms at least a portion of the underpad upper surface;
an upper tissue layer comprising a liquid permeable material, the upper tissue layer being disposed adjacent to and below a lower surface of the patient facing layer;
an absorbent layer comprising a liquid absorbing material, the absorbent layer being disposed adjacent to and below a lower surface of the tissue layer;
a lower tissue layer comprising a liquid permeable material, the lower tissue layer being disposed adjacent to and below a lower surface of the absorbent layer a base layer comprising a liquid impermeable material, the base layer being disposed below a lower surface of the absorbent layer and forming at least a portion of the underpad lower surface; and
a dye deposited on a surface of the upper tissue layer;
wherein the patient facing layer has an opacity of less than 0.80 using a diffuse opacity method (R₀/R_{∞}); and
wherein the absorbent layer has an opacity of greater than 0.80 using a diffuse opacity method (R₀/R_{∞}).

18. The underpad of claim 17 wherein the patient facing layer has an opacity of less than 0.50 using a diffuse opacity method (R₀/R_{∞}).

19. The underpad of claim 17 wherein a change in the dye is visually perceptible through the patient facing layer from the underpad upper surface subsequent to the dye being exposed to a liquid.

20. An underpad for covering a patient support structure, the underpad comprising an upper, patient facing surface and a lower, support structure facing surface, the underpad further comprising:
a patient facing layer comprising a liquid permeable material, wherein the patient facing layer forms at least a portion of the underpad upper surface;
an upper tissue layer comprising a liquid permeable material, the upper tissue layer being disposed adjacent to and below a lower surface of the patient facing layer;
an absorbent layer comprising a liquid absorbing material, the absorbent layer being disposed adjacent to and below a lower surface of the tissue layer;
a base layer comprising a liquid impermeable material, the base layer being disposed below a lower surface of the absorbent layer and forming at least a portion of the underpad lower surface; and
a dye deposited on a surface of the upper tissue layer;
wherein the dye is not visible through the patient facing layer from the underpad upper surface prior to the dye being exposed to a liquid;
wherein the dye is visible through the patient facing layer from the underpad upper surface subsequent to the dye being exposed to a liquid;
wherein the dye is less visible from the underpad lower surface; and
wherein a change in the dye is visually perceptible through the patient facing layer from the underpad upper surface subsequent to the dye being exposed to a liquid.
